(19) United States
(12) Patent Application Publication
(10) Pub. No.: US 2013/0333715 A1
(43) Pub. Date: Dec. 19, 2013

(54) SHAMPOO COMPOSITIONS AND METHODS OF MAKING SAME

(71) Applicant: The Procter & Gamble Company, Cincinnati, OH (US)

(72) Inventors: Howard David Hutton, III, Oregonia, OH (US); Jennifer Mary Marsh, Mason, OH (US)

(21) Appl. No.: 13/920,171

(22) Filed: Jun. 18, 2013

Related U.S. Application Data
(60) Provisional application No. 61/661,711, filed on Jun. 19, 2012.

(51) Int. Cl. A61K 8/88; A61Q 5/12; A61Q 5/02

(52) U.S. Cl. CPC A61K 8/88; A61Q 5/02; A61Q 5/12
USPC 132/202; 424/70.13; 424/70.17

(57) ABSTRACT

The present invention relates to a shampoo composition and methods of using the same, the composition including an ethylenediamine disuccinic acid chelant, a detersive surfactant, a cationic conditioning polymer, and a carrier.

### FIELD OF THE INVENTION

The present invention relates to a shampoo composition and methods of making and using the same. More specifically, it relates to a shampoo composition including a detersive surfactant, a cationic conditioning polymer, a chelant, and a carrier.

### BACKGROUND OF THE INVENTION

Shampoo compositions comprising various combinations of detersive surfactants, conditioning agents, and carriers are known. These products typically comprise an anionic detersive surfactant in combination with a conditioning agent such as a cationic conditioning polymer, a silicone, a hydrocarbon oil, a fatty ester, or combinations thereof. These products have become more popular among consumers as a means of conveniently obtaining hair conditioning and cleansing performance all from a single shampoo product.

Many shampoo compositions, though, do not provide sufficient deposition of conditioning agents onto hair during the cleansing process. For example, many consumers use water supplies that originate from ground water sources, which often contain various quantities of dissolved minerals such as calcite (calcium), dolomite (calcium and magnesium), magnetite (iron), and chalcanthite (copper). It has been found that even trace quantities of these minerals can deposit on the hair surface and in between the cuticle layers of hair. This deposition of minerals on hair is especially problematic for the consumers living in areas where their water source is hard, e.g., contains elevated concentrations of minerals such as calcium, magnesium, copper, and iron salts, and can lead to poor hair health. For example, deposits of calcium salts, such as calcium carbonate, between the cuticle layers can lead to poor shine and accelerate cuticle damage. Moreover, deposits of calcium salts and transition metal ions may also inhibit or interfere with the desired deposition of conditioning agents such as cationic conditioning polymers.

Without such deposition, large proportions of conditioning agent are rinsed away during the cleansing process, and, therefore, provide little or no conditioning benefit. Without sufficient deposition of the conditioning agent on the hair, relatively high levels of conditioning agents may be needed in the shampoo composition to provide adequate conditioning performance. However, high levels of a conditioning agent can increase raw material costs, reduce lathering, and present product stability concerns.

Accordingly, there is a continuing need for a shampoo composition which delivers superior conditioning benefits to hair, without a reduced cleansing performance, especially when used with hard water.

### SUMMARY OF THE INVENTION

These and other features, aspects, and advantages of the claimed invention will become evident to those skilled in the art from a reading of the present disclosure.

In accordance with an embodiment of the present invention, a shampoo composition is provided. The composition comprises a) an detersive surfactant; b) a cationic conditioning polymer; c) a chelant, and d) a carrier.

In accordance with another embodiment of the present invention, a method of reducing deposited mineral content on keratinous tissue is provided. The method comprises contacting keratinous tissue with a shampoo composition, and rinsing the shampoo composition from the keratinous tissue. The shampoo composition comprises from about 0.01 wt % to about 10 wt % of ethylene diamine disuccinic acid (EDDS) or salts thereof; from about 2 wt % to about 50 wt % of a detersive surfactant; from about 0.01 wt % to about 5 wt % of a cationic conditioning polymer; and a carrier.

### DETAILED DESCRIPTION OF THE INVENTION

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. “QS” means sufficient quantity for 100%.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights percents (wt %) as they pertain to listed ingredients are based on the active level and do not include carriers or by-products that may be included in commercially available materials, unless otherwise specified.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, and/or alcohol derivatives of a given compound.

“Polymer,” as used herein, means a chemical formed from the polymerisation of two or more monomers, which may be the same or different. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

“Kit,” as used herein, means a packaging unit comprising a plurality of components. An example of a kit is, for example, a first composition and a separately packaged second composition. Another kit may comprise a first composition and an energy delivery device. A different kit may comprise three different types of separately packaged composition and a hair styling implement. A further kit may comprise application instructions comprising a method and a composition/formulation.

The term “charge density” as used herein, means the ratio of the number of positive charges on a monomeric unit of which a polymer is comprised to the M.Wt. of said monomeric unit. The charge density multiplied by the polymer M.Wt. determines the number of positively charged sites on a given polymer chain. For cationic guars, charge density is measured using standard elemental analysis of percentage nitrogen known to one skilled in the art. This value of percentage nitrogen, corrected for total protein analysis, can then be used to calculate the number or equivalence of positive charges per gram of polymer. For the cationic copolymers, the charge density is a function of the monomers used in the synthesis. Standard NMR techniques know to one skilled in the art would be used to confirm that ratio of cationic and non-ionic monomers in the polymer. This would then be used to calculate the number or equivalence of positive charges per gram of polymer. Once these values are know, the charge density is reported in milliequivalence (meq) per gram of cationic polymer.

The term “(meth)acrylamide” as used herein means methylacrylamide or acrylamide. The term “(meth)acrylic acid” as used herein means acrylic acid or methacrylic acid.

In accordance with embodiments of the present invention, a shampoo composition is provided, the composition including an detersive surfactant, a cationic conditioning polymer, and a carrier.

The features of the composition according to the first aspect, as well as the other aspects and other relevant components, are described in detail hereinafter. All components of the composition described herein should be physically and chemically compatible with the essential components described herein, and should not otherwise unduly impair product stability, aesthetics or performance.

In accordance with one embodiment of the present invention, a shampoo composition is provided, comprising: a) a chelant; b) an detersive surfactant; c) a cationic conditioning polymer; and d) a carrier.

A. Chelants

Chelants are well known in the art and a non-exhaustive list thereof can be found in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (e.g monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

It has been found that chelants possessing a stronger affinity for redox metals (e.g., transition metal ions such as Cu⁺² and/or Fe⁺³) over that of alkaline-earth metal ions such as Ca⁺² at pH about 2 to about 6 efficiently inhibit the deposition of redox metals on keratinous, and can reduce the amount of redox metal salt deposits already existing on the keratinous tissue.

### Conditional Stability Constants of Exemplary Chelants

The relative affinity of a chelant at a specified pH for Cu⁺² versus Ca⁺² can be assessed by comparing the log of the Conditional Stability Constant of the chelant for Cu⁺² to the log of the Conditional Stability Constant of the chelant for Ca⁺² as described below.

The Conditional Stability Constant is a parameter commonly used in the art to practically assess the stability of metal-chelant complex at a given pH. A detailed discussion on Conditional Stability Constant can be found for example in “Dow chelating agents” published by the Dow Chemical Company Limited, incorporated herein by reference. The Conditional Formation Constant for a given metal referred to in this patent application is calculated using the following equation:

$${K}_{ML} ( cond ) = \frac{{K}_{ML}}{{α}_{M} \cdot {α}_{HL}}$$

$$log {K}_{ML} ( cond ) = log {K}_{ML} - log {α}_{HL} - log {α}_{M}$$

wherein K_{ML} is the Stability Constant, α_{HL} is an alpha coefficient of a partially protonated ligand (at a given pH), and α_{MOH} is an alpha coefficient of a metal hydroxide (at a given pH).

The Stability Constant of a metal chelant interaction is defined as:

$${K}_{ML} = \frac{[ ML ]}{[ M ] [ L ]}$$

where:

[ML]=concentration of metal ligand complex at equilibrium

[M]=concentration of free metal ion

[L]=concentration of free ligand in a fully deprotonated form

K_{ML}=stability constant for the metal chelant complex.

All concentrations are expressed in mol/dm³. Stability constants are conveniently expressed as logarithms. The values of the logarithms of the stability constant values for some exemplary metal ion—chelant complexes are given in the following table:

TABLE 1
Log Stability Constants for 1:1 complexes of various chelants with
Cu⁺², Fe⁺³, and Ca⁺² (fully deprotonated chelants) log K* Agent

| | log K* | | |
|---|---|---|---|
| Chelant | Cu | Fe | Ca |
| EDDS | 18.35 | 22.0 | 4.58 |
| DTPMP | 19.5 | 26.5 | 7.1 |
| EDTMP | 23.2 | 6.5 | 9.36 |
| DTPA | 21.4 | 16.4 | 10.75 |
| HEDP | 11.84 | 14.1 | 6.0 |
| EDTA | 18.78 | 14.3 | 10.65 |
| EDDHA (EHPG) | 25.3 | 35.5 | 7.2 |
| HBED | 9.29 | 39.7 | 9.3 |
| EDDG | 15.15 | * | 2.70 |
| HPDDS | 12.84 | * | 2.94 |

* All measured at 25° C. and 0.1M ionic strength
* Not available

Most chelants have a degree of protonation that is dependent on pH. This can be expressed using chelant proton stability constants (stepwise K). These stability constants are obtained from the equation below:

$$H + LHn ⇌ {LH}_{n + 1}$$

$${K}_{Hn + 1} = \frac{[ {LH}_{n + 1} ]}{[ H ] [ {LH}_{n} ]}$$

The values of the proton chelant stability constant for a few commonly known chelants are provided in Tables 2a-2d below:

TABLE 2a
Log protonation constants for tetra-dentate chelants [1]

| | HL³⁻ | H₂L²⁻ | H₃L⁻ | H₄L |
|---|---|---|---|---|
| EDDS⁴⁻ | 10.01 | 6.84 | 3.86 | 2.95 |
| HEDP⁴⁻ | 10.8 | 6.88 | 2.53 | 1.8 |
| EDTA⁴⁻ | 10.19 | 6.13 | 2.69 | 2.00 |
| EDDHA⁴⁻ | 12.1 | 9.5 | 8.5 | 6.3 |
| EDDG⁴⁻ | 9.94 | 6.50 | 4.38 | 4.23 |
| HPPDS⁴⁻ | 9.21 | 8.13 | 4.16 | 3.56 |

TABLE 2b
Log protonation constants for penta-dentate chelants [1]

| | HL⁴⁻ | H₂L³⁻ | H₃L²⁻ | H₄L⁻ | H₅L |
|---|---|---|---|---|---|
| DTPA⁵⁻ | 10.48 | 8.60 | 4.28 | 2.6 | 2.0 |

TABLE 2c
Log protonation constants for hepta-dentate chelants [1]

| | HL⁶⁻ | H₂L⁵⁻ | H₃L⁴⁻ | H₄L³⁻ | H₅L²⁻ | H₆L⁻ | H₇L |
|---|---|---|---|---|---|---|---|
| EDTMP⁷⁻ | 13.0 | 9.78 | 7.94 | 6.42 | 5.17 | 3.02 | 1.30 |

TABLE 2d
Log protonation constants for deca-protonated chelants [1]

| | HL⁹⁻ | H₂L⁸⁻ | H₃L⁷⁻ | H₄L⁶⁻ | H₅L⁵⁻ | H₆L⁴⁻ | H₇L³⁻ | H₈L²⁻ | H₉L⁻ | H₁₀L |
|---|---|---|---|---|---|---|---|---|---|---|
| DTPMP¹⁰⁻ | 11.1 | 10.1 | 8.2 | 7.2 | 6.3 | 5.5 | 4.5 | 2.8 | 2.6 | 1.5 |

[1] = Calculated using ACD Labs Version 7.0, pka calculation module (http://www.acdlabs.com/home/).

The stability constants of chelant-metal ion complexes are well documented in the literature for commonly used chelants (see for example=Arthur Martell & Robert M Smith, Critically Selected Stability Constants of Metal Complexes Database, Version 3.0 and above, incorporated herein by reference). When not documented the constants can still be measured using various analytical methods (see “Metal Complexes in Aqueous Solutions”, Martel and Hancock, edition Modem Inorganic Chemistry, p. 226-228, incorporated herein by reference).

The gradual change in ligand species as pH changes can be represented using alpha (α) coefficients, defined as:

$$alpha ( α ) coefficient ( at a given pH ) = \frac{Total concentration of ligand}{Free ligand concentration}$$

For example, in the case of tetra-acid chelants the values can be calculated from:

α_{HL}=1+K₁[H]+K₁K₂[H]²+K₁K₂K₃[H]³+K₁K₂K₃K₄[H]⁴

A further factor affecting metal chelant interactions is the tendency of metals to form hydroxide species as the pH increases. However, as the pH range of the present compositions are acidic (i.e., less than 7), the log alpha value (αₘ) is considered to be constant and approximately negligible.

By combining stability (K) and alpha (α) constants at a given pH, the formula below provides the effective chelating power of a chelant. This is the Conditional Formation Constant referred to in this patent application:

$${K}_{ML} ( cond ) = \frac{{K}_{ML}}{{α}_{M} \cdot {α}_{HL}}$$

$$log {K}_{ML} ( cond ) = log {K}_{ML} - log {α}_{HL} - log {α}_{M}$$

where, as discussed above, α_{HL} is assumed to be zero for an acid composition.

More discussion on conditional stability constant can be found, for example, in “Dow chelating agents” published by the Dow Chemical Company Limited, incorporated herein by reference. The calculated stability constants for a range of chelants with Fe⁺³, Cu⁺², and Ca⁺² are given below in Table 3:

TABLE 3
Calculated conditional stability constants for chelants with Fe⁺³,
Cu⁺², and Ca⁺² at specified pH.

| | | log Conditional Stability | | |
|---|---|---|---|---|
| | | Constant (@ pH) | | |
| Chelant | pH | Cu⁺² | Ca⁺² | Fe⁺³ |
| EDTA | 2 | 5.45 | −2.65 | |
| | 3 | 8.29 | 0.19 | |
| | 4 | 10.46 | 2.36 | |
| | 5 | 12.45 | 4.35 | |
| | 6 | 14.24 | 6.14 | |
| | 7 | 15.55 | 7.45 | |
| | 8 | 16.60 | 8.50 | |
| EDDS | 2 | 2.59 | −11.11 | 6.29 |
| | 3 | 6.28 | −7.42 | 9.98 |
| | 4 | 9.20 | −4.50 | 12.90 |
| | 5 | 11.41 | −2.29 | 15.11 |
| | 6 | 13.39 | −0.13 | 17.09 |
| | 7 | 15.06 | 1.36 | 18.76 |
| | 8 | 16.26 | 2.56 | 19.96 |
| GLD | 2 | −0.42 | −7.62 | |
| | 3 | 3.32 | −3.88 | |
| | 4 | 5.75 | −1.45 | |
| | 5 | 7.33 | 0.13 | |
| | 6 | 8.45 | 1.25 | |
| | 7 | 9.47 | 2.27 | |
| | 8 | 10.47 | 3.27 | |
| DTPA | 2 | 3.34 | −7.26 | |
| | 3 | 6.88 | −3.72 | |
| | 4 | 9.85 | −0.75 | |
| | 5 | 12.24 | 1.64 | |
| | 6 | 14.31 | 3.71 | |
| | 7 | 16.31 | 5.71 | |
| | 8 | 18.22 | 7.62 | |
| EDDHA | 2 | −3.10 | −21.20 | 7.10 |
| | 3 | 0.90 | −17.20 | 11.10 |
| | 4 | 4.90 | −13.20 | 15.10 |
| | 5 | 8.88 | −9.22 | 19.08 |
| | 6 | 12.72 | −5.38 | 22.92 |
| | 7 | 16.11 | −1.99 | 26.31 |
| | 8 | 19.07 | 0.97 | 29.27 |
| HEDTA | 2 | 3.39 | −5.81 | |
| | 3 | 6.30 | −2.90 | |
| | 4 | 8.85 | −0.35 | |
| | 5 | 10.88 | 1.68 | |
| | 6 | 12.41 | 3.21 | |
| | 7 | 13.53 | 4.33 | |
| | 8 | 14.54 | 5.34 | |
| DTPMP | 2 | −9.10 | −21.50 | −2.10 |
| | 3 | −5.10 | −17.50 | 1.90 |
| | 4 | −1.10 | −13.50 | 5.90 |
| | 5 | 2.90 | −9.50 | 9.90 |
| | 6 | 6.87 | −5.53 | 13.87 |
| | 7 | 10.68 | −1.72 | 17.68 |
| | 8 | 13.85 | 1.45 | 20.85 |
| MGDA | 2 | 3.80 | −3.10 | |
| | 3 | 5.84 | −1.06 | |
| | 4 | 7.08 | 0.18 | |
| | 5 | 8.11 | 1.21 | |
| | 6 | 9.11 | 2.21 | |
| | 7 | 10.11 | 3.21 | |
| | 8 | 11.11 | 4.21 | |
| HPPDS | 2 | −4.27 | −14.17 | |
| | 3 | −0.37 | −10.27 | |
| | 4 | 2.99 | −6.91 | |
| | 5 | 5.40 | −4.50 | |
| | 6 | 7.45 | −2.45 | |
| | 7 | 9.43 | 0.47 | |
| | 8 | 11.21 | 1.31 | |
| HBED | 2 | | | 13.54 |
| | 3 | | | 17.54 |
| | 4 | | | 21.52 |
| | 5 | | | 25.39 |
| | 6 | | | 28.82 |
| | 7 | | | 31.89 |
| | 8 | | | 34.85 |

It has been found that levels as low as about 0.01% by weight of chelants having a log K_{CaL} of less than about −2, and a log K_{CuL} of greater than about 3 or a log K_{FeL} of greater than about 10 at a pH of 5 provide acceptable inhibition of redox metal deposition, as well as an unexpected decrease in the existing redox metal deposits, on hair, wherein the log K_{CaL}, is the log of a conditional stability constant of the chelant with Ca²⁺ calculated at pH 5, the log K_{CuL} is the log of a conditional stability constant of the chelant with Cu²⁺ calculated at pH 5, and the log K_{FeL} is the log of a conditional stability constant of the chelant with Fe³⁺ calculated at pH 5. In one embodiment, a chelant having having a log K_{cal}, of less than about −2, and a log K_{CuL} of greater than about 10 or a log K_{FeL} of greater than about 15 at a pH of 5 provide acceptable inhibition of redox metal deposition, as well as an unexpected decrease in the existing redox metal deposits, on hair. The use of stability constants without taking into account the influence of the pH will give misleading results for the purpose of identifying chelants that will selectively bind to trace redox metals at low levels in hard water, and thereby inhibit the deposition of the same onto hair.

According to yet another embodiment, suitable chelants include those having log K_{CaL}/log K_{CuL} value at a pH of 5 of less 0.3. For example, the value log K_{CaL}/log K_{CuL} value at a pH of 5 may be less than 0.25, 0.20, 0.15, 0.10, 0.05, 0.00, or −0.10. In another embodiment, the chelant has a log K_{CaL}/log K_{CuL} value at a pH of 5 of about −0.2.

Accordingly, in one embodiment, the chelant is selected from diethylenetriamine penta(methylene phosphonic acid) (DTPMP); ethylenediamine-N,N′-diglutaric acid (EDDG); ethylenediamine-N,N′-bis(2-hydroxyphenylacetic acid) (EDDHA); ethylene diamine disuccinic acid (EDDS); glutamic acid diacetic acid (GLDA); hexadentate aminocarboxylate (HBED); 2-hydroxypropylendiamin-N—N′-disucinnic acid (HPDDS); methylglycinediacetic acid (MGDA); salts thereof, derivatives thereof, or mixtures thereof. Accordingly, in one embodiment, the chelant is selected from diethylenetriamine penta(methylene phosphonic acid) (DTPMP); ethylenediamine-N,N′-diglutaric acid (EDDG); ethylenediamine-N,N′-bis(2-hydroxyphenylacetic acid) (EDDHA); ethylene diamine disuccinic acid (EDDS); glutamic acid diacetic acid (GLDA); hexadentate aminocarboxylate (HBED); 2-hydroxypropylendiamin-N—N′-disucinnic acid (HPDDS); methylglycinediacetic acid (MGDA); salts and derivatives thereof, or mixtures thereof. In another embodiment, the chelant is EDDS.

Levels of such chelants in the hair care compositions can be as low as about 0.01 wt % or even as high as about 10 wt %, but above the higher level (i.e., 10 wt %) significant formulation and/or human safety concerns may arise. In an embodiment, the level of a chelant may be at least about 0.05 wt %, at least about 0.1 wt %, at least about 0.25 wt %, at least about 0.5 wt %, at least about 1 wt %, or at least about 2 wt % by weight of the hair care composition. Levels above about 4 wt % can be used but may not result in additional benefit.

B. Detersive Surfactant

According to embodiments of the present invention, the composition comprises a detersive surfactant. The detersive surfactant is included to provide cleaning performance to the composition. The detersive surfactant should be physically and chemically compatible with the essential components described herein, or should not otherwise unduly impair product stability, aesthetics, or performance. The detersive surfactant can be an anionic surfactant. In an embodiment, the shampoo composition further includes a co-surfactant, such as amphoteric surfactants, zwitterionic surfactants, cationic surfactants, non-ionic surfactants, and mixtures thereof. In an embodiment, the composition comprises from about 5 wt % to about 50 wt %, or from about 8 wt % to about 30 wt %, or from about 10 wt % to about 25 wt % of a surfactant, by total weight of the composition.

The composition may comprise a detersive surfactant system. The detersive surfactant system may comprise at least one anionic surfactant, and optionally a co-surfactant selected from the group consisting of: an amphoteric surfactant, a zwitterionic surfactant, a cationic surfactant, a nonionic surfactant, or a mixture thereof. The concentration of the detersive surfactant system in the composition should be sufficient to provide the desired cleaning and lather performance. In an embodiment, the composition comprises from about 5 wt % to about 50 wt %, or from about 8 wt % to about 30 wt %, or from about 10 wt % to about 25 wt % of detersive surfactant system, by total weight of the composition.

In considering the performance characteristics, such as coacervate formation, wet conditioning performance, dry conditioning performance, and conditioning agent deposition on hair, it is desirable to optimize the levels and types of surfactants in order to maximize the performance potential of polymer systems. In one embodiment, the detersive surfactant system for use in the composition comprises an anionic surfactant with an ethoxylate level and an anion level, wherein the ethoxylate level is from about 1 to about 10, and wherein the anion level is from about 1 to about 10. The combination of such an anionic surfactant with the cationic conditioning polymer provides enhanced deposition of the cationic conditioning polymer to hair and/or skin without reducing cleansing or lathering performance. An optimal ethoxylate level is calculated based on the stoichiometry of the surfactant structure, which in turn is based on a particular M.Wt. of the surfactant where the number of moles of ethoxylation is known. Likewise, given a specific M.Wt. of a surfactant and an anionization reaction completion measurement, the anion level can be calculated.

In an embodiment, the detersive surfactant system comprises at least one anionic surfactant comprising an anion selected from the group consisting of sulfates, sulfonates, sulfosuccinates, isethionates, carboxylates, phosphates, and phosphonates. In an embodiment, the anion is a sulfate.

In an embodiment, the anionic surfactant is an alkyl sulfate or an alkyl ether sulfate. These materials have the respective formulae ROSO₃M and RO(C₂H₄O)ₘSO₃M, wherein R is alkyl or alkenyl of from about 8 to about 18 carbon atoms, ‘m’ is an integer having a value of from about 1 to about 10, and M is a cation such as ammonium, a monovalent metal cation such as sodium and potassium, or a polyvalent metal cation such as magnesium and calcium. Solubility of the surfactant will depend upon the particular anionic surfactants and cations chosen. In one embodiment, R has from about 8 to about 18 carbon atoms, or from about 10 to about 16 carbon atoms, or from about 12 to about 14 carbon atoms, in both the alkyl sulfates and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. In an embodiment, the alcohols are lauryl alcohol and straight chain alcohols derived from coconut oil or palm kernel oil. Such alcohols are reacted with from about 0 to about 10, or from about 2 to about 5, or about 3, molar proportions of ethylene oxide, and the resulting mixture of molecular species having, for example, an average of 3 moles of ethylene oxide per mole of alcohol is sulfated and neutralized.

In an embodiment, the alkyl ether sulphate is selected from the group consisting of: sodium and ammonium salts of coconut alkyl triethylene glycol ether sulfate, tallow alkyl triethylene glycol ether sulfate, tallow alkyl hexa-oxyethylene sulphate, and mixtures thereof. In an embodiment, the alkyl ether sulfate comprises a mixture of individual compounds, wherein the compounds in the mixture have an average alkyl chain length of from about 10 to about 16 carbon atoms and an average degree of ethoxylation of from about 1 to about 4 moles of ethylene oxide. Such a mixture also comprises from about 0% to about 20% C₁₂₋₁₃ compounds; from about 60% to about 100% of C₁₄₋₁₅₋₁₆ compounds; from about 0% to about 20% by weight of C₁₂₋₁₈₋₁₉ compounds; from about 3% to about 30% by weight of compounds having a degree of ethoxylation of 0; from about 45% to about 90% by weight of compounds having a degree of ethoxylation from about 1 to about 4; from about 10% to about 25% by weight of compounds having a degree of ethoxylation from about 4 to about 8; and from about 0.1% to about 15% by weight of compounds having a degree of ethoxylation greater than about 8.

In an embodiment, the anionic surfactant is selected from the group consisting of: ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and mixtures thereof. In addition to the sulfates, isethionates, sulfonates, sulfosuccinates described above, other potential anions for the anionic surfactant include phosphonates, phosphates, and carboxylates.

The composition and/or the detersive surfactant system may comprise a co-surfactant selected from the group consisting of: amphoteric surfactants, zwitterionic surfactants, cationic surfactants, non-ionic surfactants, and mixtures thereof. The concentration of such co-surfactants may be from about 0.5% to about 20%, or from about 1% to about 10%, by total weight of the composition. In an embodiment, the composition comprises a co-surfactant selected from the group consisting of: amphoteric surfactants, zwitterionic surfactants, and mixtures thereof. Non limiting examples of suitable zwitterionic or amphoteric surfactants are described in U.S. Pat. Nos. 5,104,646 (Bolich Jr. et al.), 5,106,609 (Bolich Jr. et al.).

Amphoteric co-surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. In an embodiment, the amphoteric surfactant is selected from the group consisting of: sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof.

In one embodiment, the amphoteric co-surfactant is a surfactant according to the following structure:

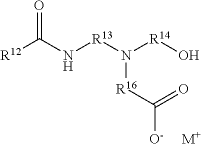

wherein R¹² is a C-linked monovalent substituent selected from the group consisting of substituted alkyl systems comprising 9 to 15 carbon atoms, unsubstituted alkyl systems comprising 9 to 15 carbon atoms, straight alkyl systems comprising 9 to 15 carbon atoms, branched alkyl systems comprising 9 to 15 carbon atoms, and unsaturated alkyl systems comprising 9 to 15 carbon atoms; R¹³, R¹⁴, and R¹⁵ are each independently selected from the group consisting of C-linked divalent straight alkyl systems comprising 1 to 3 carbon atoms, and C-linked divalent branched alkyl systems comprising 1 to 3 carbon atoms; and M⁺ is a monovalent counterion selected from the group consisting of sodium, ammonium and protonated triethanolamine. In an embodiment, the amphoteric surfactant is selected from the group consisting of: sodium cocoamphoacetate, sodium cocoamphodiacetate, sodium lauroamphoacetate, sodium lauroamphodiacetate, ammonium lauroamphoacetate, ammonium cocoamphoacetate, triethanolamine lauroamphoacetate, triethanolamine cocoamphoacetate, and mixtures thereof.

In one embodiment, the composition comprises a zwitterionic co-surfactant, wherein the zwitterionic surfactant is a derivative is a derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In an embodiment, the zwitterionic surfactant is selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof. In an embodiment, the zwitterionic surfactant is lauryl hydroxysultaine. In an embodiment, the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

In an embodiment, the co-surfactant is selected from the group consisting of: zwitterionic surfactants, amphoteric surfactants, non-ionic surfactants, and mixtures thereof. In an embodiment, the surfactant is an anionic surfactant and the composition further comprises a co-surfactant, wherein the co-surfactant is selected from the group consisting of: zwitterionic surfactants, amphoteric surfactants, non-ionic surfactants, and mixtures thereof. In an embodiment, the co-surfactant is a non-ionic surfactant selected from the group consisting of: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, and mixtures thereof. In an embodiment, the co-surfactant is a zwitterionic surfactant, wherein the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

In an embodiment, the composition comprises an anionic surfactant and a non-ionic co-surfactant. In another embodiment the surfactant system is free, or substantially free of sulfate materials. Suitable sulfate free surfactants are disclosed in WO publication 2011/120780 and WO publication 2011/049932.

C. Cationic Conditioning Polymers

According to another aspect of embodiments of the present inventions, the cationic conditioning polymer includes at least one of (a) a cationic guar polymer, (b) a cationic non-guar polymer, (c) a cationic tapioca polymer, (d) a cationic copolymer of acrylamide monomers and cationic monomers, or (e) a synthetic, non-crosslinked, cationic polymer, which forms lyotropic liquid crystals upon combination with the detersive surfactant.

(1) Cationic Guar Polymers

According to an embodiment of the present invention, the shampoo composition comprises a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide the requisite cationic charge density described above.

According to one embodiment, the cationic guar polymer has a weight average M.Wt. of less than about 1 million g/mol, and has a charge density of from about 0.1 meq/g to about 2.5 meq/g. In an embodiment, the cationic guar polymer has a weight average M.Wt. of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol from about 150 thousand to about 800 thousand g/mol, or from about 200 thousand to about 700 thousand g/mol, or from about 300 thousand to about 700 thousand g/mol, or from about 400 thousand to about 600 thousand g/mol.

In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.5 meq/g.

In an embodiment, the composition comprises from about 0.01% to less than about 0.6%, or from about 0.04% to about 0.55%, or from about 0.08% to about 0.5%, or from about 0.16% to about 0.5%, or from about 0.2% to about 0.5%, or from about 0.3% to about 0.5%, or from about 0.4% to about 0.5%, of cationic guar polymer (a), by total weight of the composition.

The cationic guar polymer may be formed from quaternary ammonium compounds. In an embodiment, the quaternary ammonium compounds for forming the cationic guar polymer conform to the general formula 1:

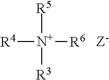

wherein where R³, R⁴ and R⁵ are methyl or ethyl groups; R⁶ is either an epoxyalkyl group of the general formula 2:

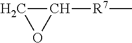

or R⁶ is a halohydrin group of the general formula 3:

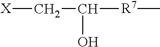

wherein R⁷ is a C₁ to C₃ alkylene; X is chlorine or bromine, and Z is an anion such as Cl—, Br—, I— or HSO₄—.

In an embodiment, the cationic guar polymer conforms to the general formula 4:

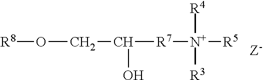

wherein R⁸ is guar gum; and wherein R⁴, R⁵, R⁶ and R⁷ are as defined above; and wherein Z is a halogen. In an embodiment, the cationic guar polymer conforms to Formula 5:

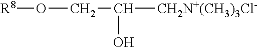

Suitable cationic guar polymers include cationic guar gum derivatives, such as guar hydroxypropyltrimonium chloride. In an embodiment, the cationic guar polymer is a guar hydroxypropyltrimonium chloride. Specific examples of guar hydroxypropyltrimonium chlorides include the Jaguar® series commercially available from Rhone-Poulenc Incorporated, for example Jaguar® C-500, commercially available from Rhodia. Jaguar® C-500 has a charge density of 0.8 meq/g and a M.Wt. of 500,000 g/mole. Another guar hydroxypropyltrimonium chloride with a charge density of about 1.1 meq/g and a M.Wt. of about 500,000 g/mole is available from Ashland. A further guar hydroxypropyltrimonium chloride with a charge density of about 1.5 meq/g and a M.Wt. of about 500,000 g/mole is available from Ashland.

Jaguar® C-17 is not suitable as the cationic guar polymer (a) for the present invention. Jaguar® C-17 conforms to Formula G and has a cationic charge density of about 0.6 meq/g and a M.Wt. of about 2.2 million g/mol and is available from Rhodia Company. Jaguar® C 13S is also not suitable for the present invention since, although it conforms to Formula G, it has a M.Wt. of 2.2 million g/mol and a cationic charge density of about 0.8 meq/g (available from Rhodia Company). In an embodiment, the present invention is substantially free of Jaguar® C-17 and/or Jaguar® C 13S.

Other suitable polymers include: Hi-Care 1000, which has a charge density of about 0.7 meq/g and a M.Wt. of about 600,000 g/mole and is available from Rhodia; N-Hance 3269 and N-Hance 3270, which have a charge density of about 0.7 meq/g and a M.Wt. of about 425,000 g/mole and is available from Ashland; AquaCat CG518 has a charge density of about 0.9 meq/g and a M.Wt. of about 50,000 g/mole and is available from Ashland.

(2) Cationic Non-Guar Polymers

The shampoo compositions of the present invention comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. Guar is an example of one type of a galactomannan polymer, specifically having a mannose to galactose ratio of 2 monomers of mannose to 1 monomer of galactose. Galactomannan polymer derivatives of the present invention have a ratio of mannose to galactose of greater than 2:1 on a monomer to monomer basis (i.e., non-guar galactomannan polymers). In one embodiment, the ratio of mannose to galactose is greater than about 3:1, and in another embodiment the ratio of mannose to galactose is greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The galactomannan polymer derivatives for use in the shampoo compositions of the present invention have a molecular weight from about 1,000 to about 10,000,000. In one embodiment of the present invention, the galactomannan polymer derivatives have a molecular weight from about 5,000 to about 3,000,000. As used herein, the term “molecular weight” refers to the weight average molecular weight. The weight average molecular weight may be measured by gel permeation chromatography.

The shampoo compositions of the present invention include galactomannan polymer derivatives which have a cationic charge density from about 0.9 meq/g to about 7 meq/g. In one embodiment of the present invention, the galactomannan polymer derivatives have a cationinc charge density from about 1 meq/g to about 5 meq/g. The degree of substitution of the cationic groups onto the galactomannan structure should be sufficient to provide the requisite cationic charge density.

In one embodiment of the present invention, the galactomannan polymer derivative is a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general formulas 1-5, as defined above.

Cationic non-guar galactomannan polymer derivatives formed from the reagents described above are represented by the general formula 6:

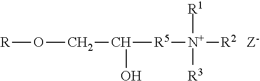

wherein R is the gum. In one embodiment, the cationic galactomannan derivative is a gum hydroxypropyltrimethylammonium chloride, which can be more specifically represented by the general formula 7:

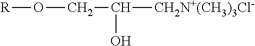

In another embodiment of the invention, the galactomannan polymer derivative is an amphoteric galactomannan polymer derivative having a net positive charge, obtained when the cationic galactomannan polymer derivative further comprises an anionic group.

The shampoo compositions of the present invention comprise at least about 0.05% of a galactomannan polymer derivative by weight of the composition. In one embodiment of the present invention, the shampoo compositions comprise from about 0.05% to about 2%, by weight of the composition, of a galactomannan polymer derivative.

(3) Cationically Modified Starch Polymer

The shampoo compositions of the present invention comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The shampoo compositions of the present invention comprise cationically modified starch polymers at a range of about 0.01% to about 10%, and in another embodiment from about 0.05% to about 5%, by weight of the composition.

The cationically modified starch polymers disclosed herein have a percent of bound nitrogen of from about 0.5% to about 4%.

The cationically modified starch polymers for use in the shampoo compositions of the present invention have a molecular weight from about 850,000 to about 15,000,000 and in one embodiment from about 900,000 to about 5,000,000. As used herein, the term “molecular weight” refers to the weight average molecular weight. The weight average molecular weight may be measured by gel permeation chromatography (“GPC”) using a Waters 600E HPLC pump and Waters 717 auto-sampler equipped with a Polymer Laboratories PL Gel MIXED-A GPC column (Part Number 1110-6200, 600.times.7.5 mm, 20 um) at a column temperature of 55.degree. C. and at a flow rate of 1.0 ml/min (mobile phase consisting of Dimethylsulfoxide with 0.1% Lithium Bromide), and using a Wyatt DAWN EOS MALLS (multi-angle laser light scattering detector) and Wyatt Optilab DSP (interferometric refractometer) detectors arranged in series (using a do/dc of 0.066), all at detector temperatures of 50° C., with a method created by using a Polymer Laboratories narrow dispersed Polysaccharide standard (Mw=47,300), with an injection volume of 200 μl.

The shampoo compositions of the present invention include cationically modified starch polymers which have a charge density from about 0.2 meq/g to about 5 meq/g, and in one embodiment from about 0.2 meq/g to about 2 meq.g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, 0. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers of the present invention generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable .sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof.

In one embodiment of the present invention, cationically modified starch polymers are selected from degraded cationic maize starch, cationic tapioca, cationic potato starch, and mixtures thereof. In another embodiment, cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers in the present invention may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance.gtoreq.80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Suitable cationically modified starch for use in compositions of the present invention is available from known starch suppliers. Also suitable for use in the present invention is nonionic modified starch that could be further derivatized to a cationically modified starch as is known in the art. Other suitable modified starch starting materials may be quaternized, as is known in the art, to produce the cationically modified starch polymer suitable for use in the invention.

Starch Degradation Procedure: In one embodiment of the present invention, a starch slurry is prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

(4) Cationic copolymer of an Acrylamide Monomer and a Cationic Monomer

According to an embodiment of the present invention, the shampoo composition comprises a cationic copolymer of an acrylamide monomer and a cationic monomer, wherein the copolymer has a charge density of from about 1.0 meq/g to about 3.0 meq/g. In an embodiment, the cationic copolymer is a synthetic cationic copolymer of acrylamide monomers and cationic monomers.

In an embodiment, the cationic copolymer comprises:
(i) an acrylamide monomer of the following Formula AM:

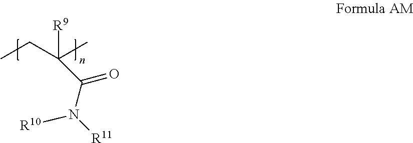

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM:

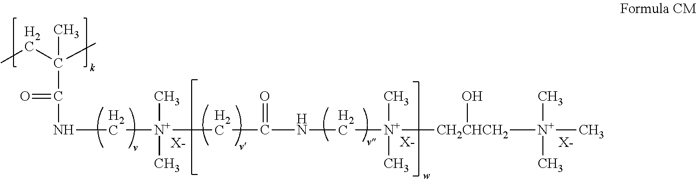

where k=1, each of v, v′, and v″ is independently an integer of from 1 to 6, w is zero or an integer of from 1 to 10, and X⁻ is an anion.

In an embodiment, cationic monomer conforming to Formula CM and where k=1, v=3 and w=0, z=1 and X⁻ is Cl⁻ to form the following structure:

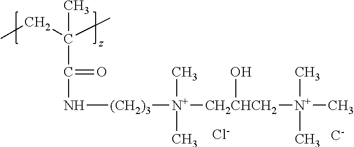

The above structure may be referred to as diquat. In another embodiment, the cationic monomer conforms to Formula CM and wherein v and v″ are each 3, v′=1, w=1, y=1 and X⁻ is Cl⁻, such as:

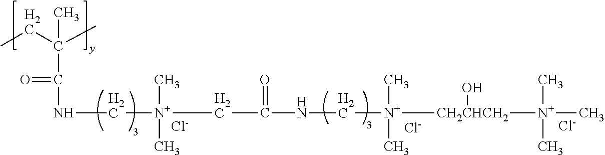

The above structure may be referred to as triquat.

In an embodiment, the acrylamide monomer is either acrylamide or methacrylamide.

In an embodiment, the cationic copolymer (b) is AM:TRIQUAT which is a copolymer of acrylamide and 1,3-Propanediaminium,N-[2-[[[dimethyl [3-[(2-methyl-1-oxo-2-propenyl)amino]propyl]ammonio]acetyl]amino]ethyl]2-hydroxy-N,N,N′,N′,N′-pentamethyl-, trichloride. AM:TRIQUAT is also known as polyquaternium 76 (PQ76). AM:TRIQUAT may have a charge density of 1.6 meq/g and a M. Wt. of 1.1 million g/mol.

In an alternative embodiment, the cationic copolymer is of an acrylamide monomer and a cationic monomer, wherein the cationic monomer is selected from the group consisting of: dimethylamino ethyl (meth)acrylate, dimethylaminopropyl (meth)acrylate, ditertiobutylamino ethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

In an embodiment, the cationic copolymer comprises a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

In an embodiment, the cationic copolymer is water-soluble. In an embodiment, the cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. In an embodiment, cationized esters of the (meth)acrylic acid containing a quaternized N atom are quaternized dialkylaminoalkyl (meth)acrylates with C1 to C3 in the alkyl and alkylene groups. In an embodiment, the cationized esters of the (meth)acrylic acid containing a quaternized N atom are selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. In an embodiment, the cationized esters of the (meth)acrylic acid containing a quaternized N atom is dimethylaminoethyl acrylate, which is quaternized with an alkyl halide, or with methyl chloride or benzyl chloride or dimethyl sulfate (ADAME-Quat). In an embodiment, the cationic monomer when based on (meth)acrylamides are quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

In an embodiment, the cationic monomer based on a (meth)acrylamide is a quaternized dialkylaminoalkyl(meth)acrylamide with C1 to C3 in the alkyl and alkylene groups. In an embodiment, the cationic monomer based on a (meth)acrylamide is dimethylaminopropylacrylamide, which is quaternized with an alkyl halide, especially methyl chloride or benzyl chloride or dimethyl sulfate.

In an embodiment, the cationic monomer is a hydrolysis-stable cationic monomer. Hydrolysis-stable cationic monomers can be, in addition to a dialkylaminoalkyl(meth)acrylamide, all monomers that can be regarded as stable to the OECD hydrolysis test. In an embodiment, the cationic monomer is hydrolysis-stable and the hydrolysis-stable cationic monomer is selected from the group consisting of: diallyldimethylammonium chloride and water-soluble, cationic styrene derivatives.

In an embodiment, the cationic copolymer is a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). In an embodiment, the cationic copolymer is formed from acrylamide and acrylamidopropyltrimethylammonium chloride, wherein the acrylamidopropyltrimethylammonium chloride has a charge density of from about 1.0 meq/g to about 3.0 meq/g.

In an embodiment, the cationic copolymer has a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

In an embodiment, the cationic copolymer has a M.Wt. from about 100 thousand g/mol to about 2 million g/mol, or from about 300 thousand g/mol to about 1.8 million g/mol, or from about 500 thousand g/mol to about 1.6 million g/mol, or from about 700 thousand g/mol to about 1.4 million g/mol, or from about 900 thousand g/mol to about 1.2 million g/mol.

In an embodiment, the cationic copolymer is a trimethylammoniopropylmethacrylamide chloride-N-Acrylamide copolymer, which is also known as AM:MAPTAC. AM:MAPTAC may have a charge density of about 1.3 meq/g and a M.Wt. of about 1.1 million g/mol. In an embodiment, the cationic copolymer is AM:ATPAC. AM:ATPAC may have a charge density of about 1.8 meq/g and a M.Wt. of about 1.1 million g/mol.

(5) Cationic Synthetic Polymer

The cationic polymer described herein aids in providing damaged hair, particularly chemically treated hair, with a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer returns the hair to a generally virgin-like, healthier state.

Lyotropic liquid crystals are formed by combining the synthetic cationic polymers described herein with the aforementioned anionic detersive surfactant component of the shampoo composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in WO 94/06403 to Reich et al. The synthetic polymers described herein can be formulated in a stable shampoo composition that provides improved conditioning performance, with respect to damaged hair. In some embodiments, the synthetic cationic polymer may be formed from

i) one or more cationic monomer units, and optionally

ii) one or more momomer units bearing a negative charge, and/or

iii) a nonionic momomer,

wherein the subsequent charge of the copolymer is positive. The ratio of the three types of monomers is given by “m”, “p” and “q” where “m” is the number of cationic monomers, “p” is the number of momomers bearing a negative charge and “q” is the number of nonionic momomers.

The concentration of the cationic polymers ranges about 0.025% to about 5%, in one embodiment from about 0.1% to about 3%, in another embodiment from about 0.2% to about 1%, by weight of the shampoo composition.

The cationic polymers have a cationic charge density of from about 2 meq/gm to about 7 meq/gm, in one embodiment from about 3 meq/gm to about 7 meq/gm, in another embodiment from about 4 meq/gm to about 7 meq/gm. In some embodiments, the cationic charge density is about 6.2 meq/gm. The polymers also have a molecular weight of from about 1,000 to about 5,000,000, in one embodiment from about 10,000 to about 2,000,000, in another embodiment 100,000 to about 2,000,000.

In one embodiment, the cationic polymers are water soluble or dispersible, non-crosslinked, synthetic cationic polymers having the following structure:

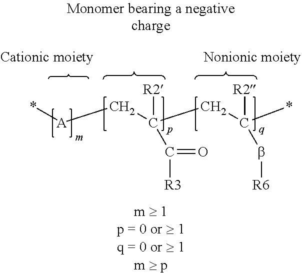

where A, may be one or more of the following cationic moieties:

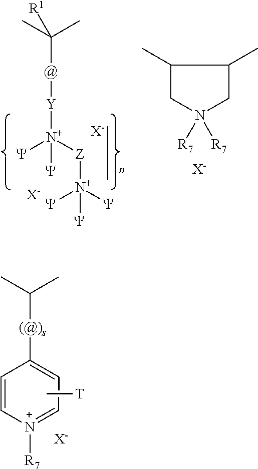

where @=amido, alkylamido, ester, ether, alkyl or alkylaryl;
where Y═C1-C22 alkyl, alkoxy, alkylidene, alkyl or aryloxy;
where w=C1-C₂₋₂ alkyl, alkyloxy, alkyl aryl or alkyl arylox;
where ψ=C1-C₂₋₂ alkyl, alkyloxy, aryl or aryloxy;
where R1=H, C1-C4 linear or branched alkyl;
where s=0 or 1, n=0 or 1;
where T and R7=C1-C22 alkyl; and
where X−=halogen, hydroxide, alkoxide, sulfate or alkylsulfate.

Where the monomer bearing a negative charge is defined by R2′=H, C1-C4 linear or branched alkyl and R3 as:

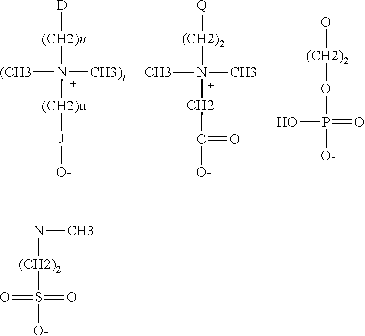

where D=O, N, or S;
where Q=NH₂ or O;
where u=1-6;
where t=0-1; and
where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

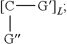

and
where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylamino ethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

In one embodiment cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

In one embodiment the cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

In one embodiment monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

In one embodiment nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X−) in association with the synthetic cationic polymers may be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non limiting examples of such counterions include halides (e.g., chlorine, fluorine, bromine, iodine), sulfate and methylsulfate.

In an embodiment, the shampoo composition comprises a plurality of cationic conditioning polymers. According to one embodiment, where two cationic conditioning polymers are present, the weight ratio of a first cationic conditioning polymer to a second cationic conditioning polymer is from about 1000:1 to about 2:1. In an embodiment, the weight ratio of the first cationic conditioning polymer to the second cationic conditioning polymer is from about 1000:1 to about 4:1. In an embodiment, weight ratio of the first cationic conditioning polymer to the second cationic conditioning polymer is from about 800:1 to about 4:1, or from about 500:1 to about 4:1, or from about 100:1 to about 5:1, or from about 100:1 to about 6:1, or from about 50:1 to about 6.5:1, or from about 50:1 to about 7:1, or from about 50:1 to about 8.3:1, or from about 50:1 to about 16.7:1.

D. Carrier

In accordance with another embodiment, the composition further comprises a cosmetically acceptable carrier. In an embodiment, the carrier is an aqueous carrier. The amount and chemistry of the carrier is selected according to the compatibility with other components and other desired characteristic of the product. In an embodiment, the carrier is selected from the group consisting of: water and water solutions of lower alkyl alcohols. Lower alkyl alcohols useful herein are monohydric alcohols having 1 to 6 carbons, such as ethanol and/or isopropanol. In an embodiment, the cosmetically acceptable carrier is a cosmetically acceptable aqueous carrier and is present at a level of from about 20% to about 95%, or from about 60% to about 85%.

The pH composition may be from about pH 3 to about pH 9, or from about pH 4 to about pH 7.

E. Benefit Agent

In accordance with embodiments of the present invention, the shampoo composition may further comprise one or more benefit agents. Exemplary benefit agents include, but are not limited to, silicone emulsions, anti-dandruff actives, perfume microcapsules, gel networks, colorants, particles, and other insoluble skin or hair conditioning agents such as skin silicones, natural oils such as sun flower oil or castor oil.

(1). Silicone Emulsion

The silicone emulsions suitable for use in the embodiments of the present invention include emulsions of insoluble polysiloxanes prepared in accordance with the descriptions provided in U.S. Pat. No. 4,476,282 and U.S. patent application Publication No. 2007/0276087. Accordingly, insoluble polysiloxanes referred to herein for the purpose of the invention include polysiloxanes such as alpha, omega hydroxy-terminated polysiloxanes or alpha, omega alkoxy-terminated polysiloxanes having a molecular weight within the range from about 50,000 to about 500,000 g/mol. As used herein, “insoluble polysiloxane” means that the water solubility of the polysiloxane is less than 0.05 wt %. In another embodiment, the water solubility of the polysiloxane is less than 0.02 wt %, or less than 0.01 wt %, or less than 0.001 wt %. According to an embodiment, the insoluble polysiloxane is present in the shampoo composition in an amount within the range from about 0.1 wt % to about 3 wt %, based on the total weight of the composition. For example, the insoluble polysiloxane can be present in an amount within the range from about 0.2 wt % to about 2.5 wt %, or from about 0.4 wt % to about 2.0 wt %, or from about 0.5 wt % to about 1.5 wt %, based on the total weight of the composition.

According to one aspect of the silicone emulsion, the insoluble polysiloxane used herein include alpha, omega hydroxy- or alkoxy-terminated polysiloxanes having a general formula I:

R¹—[O—SiR₂]ₙ—OR¹,

wherein ‘n’ is an integer, R is a substituted or unsubstituted C₁ to C₁₀ alkyl or aryl, and R¹ is a hydrogen or a substituted or unsubstituted C₁ to C₁₀ alkyl or aryl. Non-limiting examples of R and R¹ may be independently selected from alkyl groups such as methyl, ethyl, n-propyl, isopropyl, n-butyl, isobutyl, tert-butyl, n-pentyl, isopentyl, neopentyl, tertpentyl, hexyl such as n-hexyl, heptyl such as n-heptyl, octyl such as n-octyl and isooctyl such as 2,2,4-trimethyl-pentyl, nonyl such as n-nonyl, decyl such as n-decyl, dodecyl such as n-dodecyl, octadecyl such as n-octadecyl; or aryl groups such as phenyl, naphthyl, anthryl and phenanthryl. In an embodiment, the insoluble polysiloxane has a general formula H—[O—SiR₂]ₙ—OH.

According to another aspect of the silicone emulsion, the insoluble polysiloxane has an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol.

According to another aspect of the silicon emulsion, total content of a cyclic polysiloxane having a general formula:

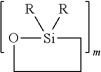

wherein R is as defined above, and wherein m is 4 or 5, is present in the silicone emulsion in an amount less than about 2.5 wt % based on the total weight of all polysiloxanes. For example, dimethiconol may include significant quantities of cyclic polysiloxanes, such as octamethylcyclotetrasiloxane (D4) and decamethylcyclotetrasiloxane (D5). In an embodiment, the amount of D4 is less than about 2.0%, or less than about 1.5%, or less than about 1.0%, or less than about 0.5%, based on the total weight of all polysiloxanes. In an embodiment, the amount of D5 is less than about 0.5%, or less than about 0.4%, or less than about 0.3%, or less than about 0.2%, based on the total weight of all polysiloxanes.

According to yet another aspect of the silicone emulsion, the emulsion has a viscosity up to about 500,000 cPs. For example, the viscosity may be within the range from about 75,000 to about 300,000, from about 100,000 to about 200,000, or about 150,000 cPs.

According to yet another aspect of the silicone emulsion, the insoluble polysiloxane has an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The average molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscosimeter with spindle 6 at 2.5 rpm.

According to another aspect of the silicone emulsion, the emulsion further includes an anionic surfactant that participates in providing high internal phase viscosity emulsions having particle sizes in the range from about 30 nm to about 10 micron. The anionic surfactant is selected from organic sulfonic acids. Most common sulfonic acids used in the present process are alkylaryl sulfonic acid; alkylaryl polyoxyethylene sulphonic acid; alkyl sulfonic acid; and alkyl polyoxyethylene sulfonic acid. General formulas of the sulfonic acids are as shown below:

R²C₆H₄SO₃H (II)

R²C₆H₄O(C₂H₄O)ₘSO₃H (III)

R²SO₃H (IV)

R²O(C₂H₄O)ₘSO₃H (IV)

Where R², which may differ, is a monovalent hydrocarbon radical having at least 6 carbon atoms. Non-limiting examples of R² include hexyl, octyl, decyl, dodecyl, cetyl, stearyl, myristyl, and oleyl. ‘m’ is an integer from 1 to 25. Exemplary anionic surfactants include but are not limited to octylbenzene sulfonic acid; dodecylbenzene sulfonic acid; cetylbenzene sulfonic acid; alpha-octyl sulfonic acid; alpha-dodecyl sulfonic acid; alpha-cetyl sulfonic acid; polyoxyethylene octylbenzene sulfonic acid; polyoxyethylene dodecylbenzene sulfonic acid; polyoxyethylene cetylbenzene sulfonic acid; polyoxyethylene octyl sulfonic acid; polyoxyethylene dodecyl sulfonic acid; and polyoxyethylene cetyl sulfonic acid. Generally, 1 to 15% anionic surfactant is used in the emulsion process. For example, 3-10% anionic surfactant can be used to obtain an optimum result.

The silicone emulsion may further include an additional emulsifier together with the anionic surfactant, which along with the controlled temperature of emulsification and polymerization, facilitates making the emulsion in a simple and faster way. Non-ionic emulsifiers having a hydrophilic lipophilic balance (HLB) value of 10 to 19 are suitable and include polyoxyalkylene alkyl ether, polyoxyalkylene alkylphenyl ethers and polyoxyalkylene sorbitan esters. Some useful emulsifiers having an HLB value of 10 to 19 include, but are not limited to, polyethylene glycol octyl ether; polyethylene glycol lauryl ether; polyethylene glycol tridecyl ether; polyethylene glycol cetyl ether; polyethylene glycol stearyl ether; polyethylene glycol nonylphenyl ether; polyethylene glycol dodecylphenyl ether; polyethylene glycol cetylphenyl ether; polyethylene glycol stearylphenyl ether; polyethylene glycol sorbitan monostearate; and polyethylene glycol sorbitan monooleate.

In accordance with another embodiment, the composition may further comprise an anti-dandruff active, which may be an anti-dandruff active particulate.

F. Other Components

The shampoo compositions of the present invention can also additionally comprise any suitable optional ingredients as desired. Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

In accordance with another embodiment of the invention, a method of making a shampoo composition comprising a detersive surfactant, a cationic conditioning polymer, a chelant, and a carrier is provided. The method includes (i) combining the detersive surfactant and the cationic conditioning polymer in suitable carrier, and (ii) combining a chelant and a carrier composition that includes a product of step (i) to form the shampoo composition.

In an embodiment, the shampoo composition has a viscosity of 4,000 cP to 20,000 cP, or from about 6,000 cP to about 12,000 cP, or from about 8,000 cP to about 11,000 cP, measured at 26.6° C. with a Brookfield R/S Plus Rheometer at 2 s⁻¹. cP means centipoises.

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

### EXAMPLES

Exemplary shampoo compositions, in accordance with the principles of this disclosure, can be prepared as set forth in Table 1.

| Ingredient | A | B | C | D | E | F | G |
|---|---|---|---|---|---|---|---|
| sodium lauryl ether sulfate (SLE3S [symbol] | | 6 | 10 | 6 | 6 | 9 | |
| Sodium cocoyl isethionate | | | | | | | 8.5 |
| sodium lauryl sulfate (SLS) | 1.5 | 7 | 1.5 | 7 | 7 | 6 | |
| sodium lauryl ether sulfate (SLE1S [symbol] | 10.5 | | | | | | |
| Disodium laureth sulfosuccinate | | | | | | | 8.5 |
| Sodium lauryl sulfoacetate | | | | | | | 2.5 |
| Sodium Lauroyl Sarcosinate | | | | | | | 0.75 |
| Cocoamidopropyl Hydroxysultaine | | | | | | | 1.5 |
| Cocoamidopropyl Betaine | | | | | | | 2 |
| cocamidopropyl betaine (CapB) | 1 | 2 | 2 | 2 | 2 | 2 | |
| Coconut monoethanol amide (CMEA) | | 0.85 | | 0.85 | | | |
| Gel Network (C16OH/C18OH) | | | 2 | | | | |
| dimethicone | 1 | 1 | 1 | 1 | 1 | | 0.5 |
| Ethylene glycol distearate (EGDS) | 1.5 | 1.5 | 1.5 | 1.5 | 1.5 | | |
| Jaguar ® C500 | 0.25 | 0.25 | 0.15 | | | | |
| Synthetic Cationic Polymer AMT | | | | 0.1 | | | |
| Synthetic Cationic Polymer DADMAC | | | | | 0.1 | | |
| Excel Guar | | | | | | 0.1 | .15 |
| Ethylene Diamine Disuccinic acid (EDDS) | 0.1, 0.5 | 0.1, 0.5 | 0.1, 0.5 | 0.1, 0.5 | 0.1, 0.5 | 0.1, 0.5 | |
| pH | 6 | 6 | 6 | 6 | 6 | 6 | |
| Water-USP Purified & Minors | Q.S. | Q.S. | Q.S. | Q.S. | Q.S. | Q.S. | Q.S. |
| | to 100 | to 100 | to 10 [symbol] | to 10 [symbol] | to 10 [symbol] | to 10 [symbol] | to 10 [symbol] |

[symbol]

indicates data missing or illegible when filed

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shampoo composition comprising:
a. from about 0.01 wt % to about 10 wt % of ethylene diamine disuccinic acid (EDDS) or salts thereof;
b. from about 2 wt % to about 50 wt % of a detersive surfactant;
c. from about 0.01 wt % to about 5 wt % of a cationic conditioning polymer; and
d. a carrier.

2. The shampoo composition of claim 1, wherein the cationic conditioning polymer is a cationic galactomannon polymer derivative.

3. The shampoo composition of claim 2, wherein the cationic galactomannan polymer derivative is a non-guar cationic polymer.

4. The shampoo composition of claim 1, wherein the cationic conditioning polymer is a cationic modified starch.

5. The shampoo composition of claim 1, wherein the cationic condition polymer is a synthetic random copolymer having a net positive charge comprising:
(i) an acrylamide monomer of the following Formula AM: 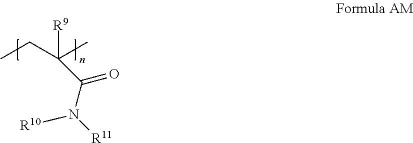
where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM: 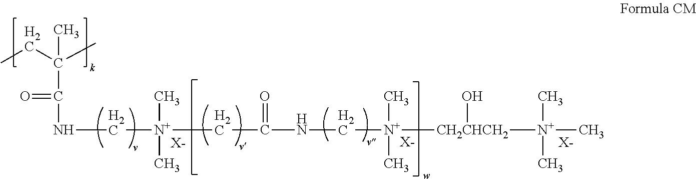
where k=1, each of v, v′, and v″ is independently an integer of from 1 to 6, w is zero or an integer of from 1 to 10, and X⁻ is an anion.

6. The shampoo composition of claim 1, wherein the cationic conditioning polymer is a synthetic, non-crosslinked, cationic polymer having a cationic charge density of from about 2 meq/gm to about 7 meq/gm, and wherein said synthetic, non-crosslinked, cationic polymer forms lyotropic liquid crystals upon combination with said detersive surfactant.

7. In one embodiment, the cationic polymers are water soluble or dispersible, non-crosslinked, synthetic cationic polymers having the following structure: 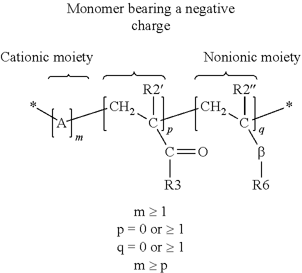
where A, may be one or more of the following cationic moieties: 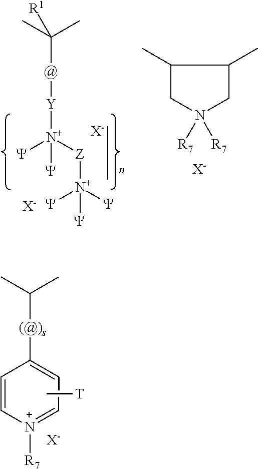
12.
where @=amido, alkylamido, ester, ether, alkyl or alkylaryl;
where Y═C₁-C₂₂ alkyl, alkoxy, alkylidene, alkyl or aryloxy;
where ψ=C₁-C₂₂ alkyl, alkyloxy, alkyl aryl or alkyl aryloxy;
where Z=C1-C₂₂ alkyl, alkyloxy, aryl or aryloxy;
where R1=H, C1-C4 linear or branched alkyl;
where s=0 or 1, n=0 or ≧1;
where T and R7=C1-C22 alkyl;
where X—=halogen, hydroxide, alkoxide, sulfate or alkylsulfate;
where the monomer bearing a negative charge is defined by R2′═H, C1-C4 linear or branched alkyl and R³ as: 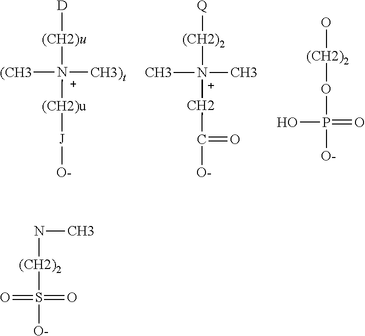
where D=O, N, or S;
where Q=NH₂ or O;
where u=1-6;
where t=0-1;
where J=oxygenated functional group containing the following elements P, S, C;
where the nonionic monomer is defined by R²″=H, C1-C4 linear or branched alkyl, R⁶=linear or branched alkyl, alkyl aryl, aryl oxy, alkyloxy, alkylaryl oxy and β is defined as 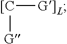
and
where G′ and G″ are, independently of one another, O, S or N—H and L=0 or 1.

8. The shampoo composition of claim 1, wherein said synthetic, non-crosslinked, cationic polymer comprises monomers selected from the group consisting of dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride and trimethyl ammonium propyl (meth)acrylamido chloride.

9. The shampoo composition of claim 1, wherein the detersive surfactant is selected from an anionic surfactant, cationic surfactant, non-ionic surfactant, amphoteric surfactants, or mixtures thereof.

10. The shampoo composition of claim 1, wherein the detersive surfactant is present in an amount ranging from about 5 wt % to about 25 wt %.

11. The shampoo composition of claim 1, further comprising a gel network comprising a fatty alcohol and a surfactant.

12. A method of reducing deposited mineral content on keratinous tissue, comprising:
1) contacting keratinous tissue with a shampoo composition comprising:
a. from about 0.01 wt % to about 10 wt % of ethylene diamine disuccinic acid (EDDS) or salts thereof;
b. from about 2 wt % to about 50 wt % of a detersive surfactant;
c. from about 0.01 wt % to about 5 wt % of a cationic conditioning polymer or copolymer; and
d. a carrier; and
2) rinsing the shampoo composition from the keratinous tissue.

13. The method of claim 12, wherein the cationic conditioning polymer is a cationic galactomannon polymer derivative.

14. The method of claim 12, wherein the cationic galactomannan polymer derivative is a non-guar cationic polymer.

15. The method of claim 12, wherein the cationic conditioning polymer is a cationic modified starch.

16. The method of claim 12, wherein the cationic condition polymer is a synthetic random copolymer having a net positive charge comprising:
(i) an acrylamide monomer of the following Formula AM: 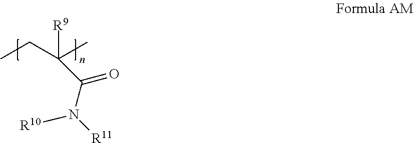
where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM: 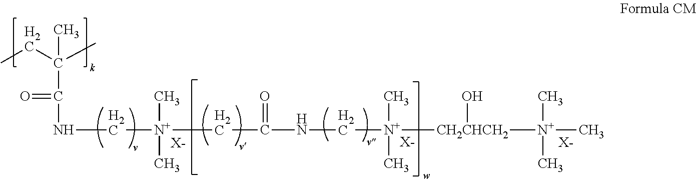
where k=1, each of v, v′, and v″ is independently an integer of from 1 to 6, w is zero or an integer of from 1 to 10, and X⁻ is an anion.

17. The method of claim 12, wherein the cationic condition polymer is a synthetic, non-crosslinked, cationic polymer having a cationic charge density of from about 2 meq/gm to about 7 meq/gm, and wherein said synthetic, non-crosslinked, cationic polymer forms lyotropic liquid crystals upon combination with said detersive surfactant.
